(19) United States
(12) Patent Application Publication
(10) Pub. No.: US 2026/0057209 A1
(43) Pub. Date: Feb. 26, 2026

(54) Resource Allocation and Treatment Recommendations for Hemorrhage Casualties Method and System

(71) Applicants: The Government of the United States, as Represented by the Director of the Defense Health Agency, Fort Detrick, MD (US); Henry M. Jackson Foundation for the Advancement of Military Medicine, Inc., Bethesda, MD (US)

(72) Inventors: Jaques Reifman, New Market, MD (US); Sven Anders Wallqvist, Frederick, MD (US); Xin Jin, Frederick, MD (US)

(21) Appl. No.: 19/307,784

(22) Filed: Aug. 22, 2025

Related U.S. Application Data
(60) Provisional application No. 63/686,847, filed on Aug. 25, 2024.

(51) Int. Cl. G06N 3/0442; A61B 5/00; A61B 5/0205; A61B 5/021; A61B 5/024; G06N 3/0499

(52) U.S. Cl. CPC G06N 3/0442; A61B 5/02055; A61B 5/021; A61B 5/024; A61B 5/48; G06N 3/0499

(57) ABSTRACT

A model was developed to predict vital-signs of hemorrhage patients and optimize the management of fluid resuscitation in mass casualties. In at least one embodiment, the model uses a limited data stream (the initial 10 minutes of vital-sign monitoring) to predict at an individual (personalized) level the outcomes of different fluid resuscitation allocations 60 minutes into the future. The predicted outcomes were then used to select the optimal resuscitation allocation for various simulated mass-casualty scenarios. The theoretical benefits of this approach included up to 46% additional casualties restored to healthy vital signs and a 119% increase in fluid-utilization efficiency. The greatest benefit of this technology lies in its ability to provide personalized interventions that optimize clinical outcomes under resource-limited conditions, such as in civilian or military mass-casualty events, involving moderate and severe hemorrhage.

This patent application claims the benefit of U.S. Patent Application No. 63/686,847, filed on Aug. 25, 2024, which is hereby incorporated by reference.

### I. FIELD OF THE INVENTION

The ability to identify and treat hemorrhage continues to be a top priority in combat casualty care, and attention is increasingly shifting toward conditions involving multiple casualties in austere and resource-constrained environments. To address this problem, a recurrent neural network-based artificial intelligence (AI) model was developed that predicts vital signs of hemorrhage patients and optimizes the management of fluid resuscitation in mass casualties to maximize the number of casualties returned to a stable condition.

### II. BACKGROUND

Uncontrolled bleeding remains the major cause of preventable civilian and battlefield trauma deaths, with the greatest loss of life occurring in the pre-hospital environment. The ability to identify and treat hemorrhage continues to be a top priority in combat casualty care, and attention is increasingly shifting toward conditions involving multiple casualties in austere and resource-constrained environments. In these cases, AI technologies represent a promising approach, providing decision support for triage, treatment, and resource prioritization at the lowest echelons of care (Raita et al., 2019; Fernandes et al., 2020; Liu et al., 2023; Peng et al., 2023; Stallings et al., 2023).

The U.S. Department of Defense (DoD) has established practical population-based guidelines, procedures, and protocols to help combat medics identify and treat trauma-induced hemorrhage and provide fluid resuscitation in accordance with current vital signs and symptoms, such as those provided by the Vampire Program (Voller et al., 2021) and the Tactical Combat Casualty Care guidelines (Deaton et al., 2021). These protocols include robust and tried procedures that optimize outcomes when resources are readily available. Although they are based on population studies and do not provide patient-specific recommendations, these guidelines represent the current standard of care, designed to support trained medics. However, these approaches may lead to over or under use of resuscitation fluids that will impact the opportunity to have improved outcomes for more individuals in a mass casualty situation.

Machine-learning methods have been proposed to support the automation of casualty treatment, such as closed-loop fluid resuscitation (Kramer et al., 2008; Rinehart et al., 2013; Marques et al., 2017; Jin et al., 2018; Gholami et al., 2021; Alsalti et al., 2022; Avital et al., 2022). Although these treatments can potentially optimize fluid resuscitation for one casualty at a time, they do not address the simultaneous management of multiple casualties under resource-constrained conditions. Similarly, for the unstructured pre-hospital and the structured hospital environments, machine-learning methods have been developed to predict the need for life-saving interventions (Liu et al., 2014), including massive blood transfusion (Mina et al., 2013; Hodgman et al., 2018; Lammers et al., 2022), and to automatically analyze vital-sign data and stratify hemorrhage risk in trauma casualties (Stallings et al., 2023). While these methods flag the need for treatment, they do not necessarily provide a personalized resuscitation plan for each individual casualty.

Therefore, a need still exists for an approach to maximize the use of resuscitation fluid in a mass casualty situation where there may be limited supplies and minimal opportunity for resupply or evacuation of any casualty.

### III. SUMMARY

The development, integration, and application of AI and machine-learning methods in medical care will be critical in future military conflicts to overcome anticipated challenges associated with high casualty rates, medical evacuation delays, and prolonged field care with limited resources. At all roles of medical care, the caregiver will need to efficiently match injuries with appropriate treatments under stressful conditions and with variable resource availability. It may be possible that the caregiver is not a medic or medically trained and/or sufficiently cognizant of the situation (e.g., sleep deprived and/or overwhelm). A recurrent artificial neural network model was trained on time-series vital-sign data to predict trauma-induced hemorrhage outcomes and use this knowledge to optimize casualty treatment under resource-limited conditions.

Arguably, the main factor limiting the development of data-driven AI solutions for field care and triage of hemorrhage injuries is the lack of well-annotated and curated data to train these algorithms. In particular, artificial neural network approaches used in the most powerful applications, such as large language models in medicine (Walker et al., 2023), require depth and breadth of quality data to become reliable. Here, this problem was addressed by using synthetic data derived from a previously developed cardiovascular-respiratory (CR) mathematical model (Jin et al., 2023) to simulate multiple moderate to severe hemorrhage and fluid resuscitation scenarios, which were used to develop a recurrent artificial neural network model capable of predicting treatment outcomes for each casualty 60 minutes into the future based on just 10 minutes of vital-sign data as input to the AI model to personalize the model.

As relevant clinical data of moderate to severe hemorrhage in the pre-hospital environment are scarce and insufficient for training AI models, the use of synthetic data is key in developing and understanding the behavior of such data-driven approaches. Hence, the CR model was used to generate synthetic data representing the time evolution of vital signs after the onset of hemorrhage, followed by the application of a tourniquet and subsequent transfusion of resuscitation fluid. By varying the volume of blood loss and transfused fluid as well as the timing of events and model parameters, the CR model allowed the generation of a sufficiently large number of synthetic casualties that were used to train a recurrent neural network AI model. In addition, the computational CR model also served as the ground-truth physiological response for human casualties against which the model predictions were compared. In particular, the AI model allowed exploration of optimizing fluid allocations under resource-limited conditions for compressible bleeding and detecting uncontrolled non-compressible bleeding.

More particularly, using the CR model to generate synthetic data of hemorrhage casualties, a model was developed where a limited data stream (the initial 10 minutes of vital-sign monitoring) could be used to personalize and predict the outcomes of different fluid resuscitation allocations 60 minutes into the future. The knowledge of the predicted outcomes was used to select the optimal resuscitation allocation for simulated mass-casualty scenarios.

The model was trained with simulated vital-sign data (heart rate and blood pressure) for hemorrhage in an extremity for ten minutes prior to application of a tourniquet and for sixty minutes after application of the tourniquet with four treatment options (no resuscitation fluid, one unit of resuscitation fluid in either the first 30 minutes or the second 30 minutes, and two units of resuscitation fluid).

Although the CR model represents a simplified hemodynamic cardiovascular response, it does capture the correct coupled physiological behavior of heart rate and systolic blood pressure variation during hemorrhage and fluid resuscitation. This allowed assessment of the potential benefits of using an allocation method that is based on personalized predictions of future outcomes versus a static population-based method that only uses currently available vital-sign information. The resource-allocation method that used the AI-predicted outcomes was contrasted with the DoD's Vampire Program to understand the prospective value and benefits of using such a method in mass-casualty scenarios and various resource-limited conditions. The theoretical benefits of this approach based on computer simulations include up to 46% additional casualties restored to healthy vital signs and up to a 119% efficiency increase in fluid utilization as discussed in the article.

The work demonstrated the potential for incorporating artificial neural network-based technologies in hemorrhage treatment. The simulated injury and treatment scenarios used delineated possible benefits and opportunities available for using AI in pre-hospital trauma care. The greatest benefit of this technology lies in its ability to provide personalized interventions that optimize clinical outcomes under resource-limited conditions, such as in civilian or military mass-casualty events, involving moderate and severe hemorrhage.

In at least one embodiment, the model includes an input layer, a first feedforward layer, a gated recurrent (GRU) layer, a second feedforward layer, and an output layer consisting of the projected heart rate and systolic blood pressure.

In a further embodiment, after the input layer, each node in the first feedforward layer receives all three inputs and outputs to all of the nodes of the GRU layer. Each node in the GRU layer outputs to each node in the second feedforward layer and back to itself (i.e., to all its own nodes). The nodes of the second feedforward layer output to the output layer, which together project heart rate and systolic blood pressure at a future time, which then is feed back into the model to obtain the projection for subsequent heart rate and systolic blood pressure values, iteratively reaching 30 minutes and 60 minutes out from the present time for each of the possible infusion regiments to determine which regiment(s) returns the casualty back to healthy and stable vital sign levels. In a further embodiment to any of the other embodiments, each feedforward or GRU layer includes 512 nodes. In a further embodiment to any of the other embodiments, 8-10 minutes of heart rate and systolic blood pressure measurements are used to personalize the model.

In at least one embodiment, for each casualty being monitored, a different instance of the model is used and the system includes a dashboard interface that displays the different modal instances running for each of the casualties including vital sign information and treatment recommendation (i.e., resuscitation fluid allocation). The dashboard displays the amount of fluid units available, used, and held in reserve, for example for a casualty that requires one unit in the future. The model uses the number of instances running and in at least one embodiment, the level of resuscitation fluid required to balance the use of the existing supply of resuscitation fluid. For example, if a large percentage of a unit has been wounded and there is minimal risk for additional casualties, then the model will be more aggressive in the administration of resuscitation fluid including providing more than one unit if recommended. Conversely if there is a risk for more casualties, then the model will be more conservative and provide recommendations to conserve the use of resuscitation fluid and minimize the allocation of two units.

In at least one method embodiment for allocating of resuscitation fluid to one or more casualties, the method includes: collecting multiple minutes of vital-sign data from vital-sign sensors attached to one casualty, applying hemorrhage control to stop or largely stop the bleeding of the casualty, inputting the collected vital-sign data into a model to generate a personalized predicted set of vital signs for the casualty after running multiple scenarios, wherein each scenario is associated with an amount and an infusion rate of resuscitation fluid to be provided to the casualty over a predetermined time period, selecting the scenario that uses the least amount of resuscitation fluid to have, if possible, the casualty vital signs within healthy target ranges at the end of the predetermined time period while ensuring a largest number of casualties reach healthy target ranges, and administrating resuscitation fluid to the casualty pursuant to the selected scenario; and wherein the vital-sign data includes a heart rate and a systolic blood pressure, and the infusion rate for resuscitation fluid is a third input into the model.

In at least one method embodiment for allocating of resuscitation fluid to one or more casualties, the method includes: for each casualty, collecting multiple minutes of vital-sign data from vital-sign sensors attached to the casualty, where the vital-sign data includes a heart rate and a systolic blood pressure, applying hemorrhage control to stop or largely stop the bleeding of the casualty, inputting the collected vital-sign data into a model to generate a personalized predicted set of vital signs for the casualty after running multiple scenarios, wherein each scenario is associated with an amount and an infusion rate of resuscitation fluid to be provided to the casualty over a predetermined time period, displaying to a dashboard a recommended scenario that uses the least amount of resuscitation fluid to have, if possible, the casualty vital signs within healthy target ranges at the end of the predetermined time period, and administrating resuscitation fluid to the casualty pursuant to the recommended scenario; and wherein the dashboard that includes information for each casualty including when to administer resuscitation fluid to each casualty considering a supply level of resuscitation fluid.

In a further method embodiment to the above method embodiments, the predetermined time period is sixty minutes and the multiple scenarios include the following four scenarios for sixty minutes after application of the hemorrhage control: providing the casualty no resuscitation fluid, providing the casualty one unit of resuscitation fluid in either the first 30 minutes or the second 30 minutes, and providing the casualty two units of resuscitation fluid with one unit in the first 30 minutes and the second unit in the second 30 minutes. In a further method embodiment to the above method embodiments, the resuscitation fluid is whole blood, blood products, saline, or crystalloids. In a further method embodiment to the above method embodiments, the vital sign data is collected for at least 10 minutes and the predetermined time period is sixty minutes. In a further method embodiment to the above method embodiments, the hemorrhage control is a tourniquet.

In a further method embodiment to the above method embodiments, the model is a recurrent artificial neural network. In a further embodiment, the model includes a series of layers between an input layer and an output layer, where each layer includes 128, 256, or 512 nodes. In a further embodiment to the other embodiments of this paragraph, the model includes an input layer, a first feedforward layer, a gated recurrent unit (GRU) layer, a second feedforward layer, and an output layer where the outputs of the first feedforward layer is a n-dimensional feature vector that is inputted into the GRU layer, the outputs of the GRU layer is a m-dimensional feature vector that is inputted into the GRU layer and the second feedforward layer, and the outputs of the second feedforward layer is a o-dimensional feature vector that in inputted in the output layer that provides the outputs for the model that are then inputted back into the model, and wherein n, m, and o are equal to the number of nodes of the respective layer.

In a further method embodiment to the above method embodiments, wherein inputting the collected vital sign data into the model includes receiving the heart rate at a given time (“HR(t)”), the fluid infusion rate at said given time (“uₜ(t)”), and the systolic blood pressure at said given time (“SBP(t)”) into an input layer that distributes the inputs to each node of a first feedforward layer, the first feedforward layer having a plurality of first feedforward layer nodes; receiving a first dimensional feature vector produced by the first feedforward layer into a gated recurrent unit layer (“GRU”) having a plurality of GRU layer nodes for personalizing the prediction for the casualty based on the inputs into the GRU layer, and the GRU layer further receiving a second dimensional feature vector from the GRU layer previous time period on a second iteration of the method through the model; receiving the second dimensional feature vector from the GRU layer into a second feedforward layer having a plurality of second feedforward layer nodes, where each second feedforward layer node receives the second dimensional feature vector; receiving a third dimensional vector produced by the second feedforward layer into an output layer; outputting a personalized predicted value of the heart rate at a future time (“HR(t+1)”) and a predicted value of systolic blood pressure at a future time (“SBP(t+1)”) from the output layer, and wherein the method is repeatedly iterated for the predetermined time period, and the predetermined time period is sixty minutes, and during a subsequent iteration of the method, the inputs sent to the nodes of the input layer and then to the first feedforward layer are the HR(t+1) and SBP(t+1) output from a prior iteration of the method.

In a further method embodiment to the above method embodiments, the model is trained on vital signs generated by a cardio-respiratory mathematical model to simulate future heart rate and systolic blood pressure based on inputted heart rates, systolic blood pressures, and infusion rates. In a further method embodiment to the above method embodiments, the model includes at least one hidden state, and utilizes measured vital sign data to update the at least one hidden state to personalize the model for the casualty, and starting at application of hemorrhage control, the model continuously predicts the vital sign data for each subsequent minute using the infusion rate associated with one of the scenarios and feeding back into the model the calculated vital signs until the model predicts the vital sign data at the end of the predetermined time period to allow for a comparison between the multiple scenarios.

In at least one system embodiment for providing a recommendation for use of resuscitation fluid in hemorrhage treatment for multiple casualties, the system includes: vital sign sensors configured to be attached to each casualty, the vital sign sensors include a heart rate monitor and a systolic blood pressure sensor; a memory; a processor having a model trained on hemorrhage control and resuscitation fluid recovery scenarios, the model configured to provide a recommendation regarding use of resuscitation fluid once hemorrhage control has begun on the casualty, the processor in communication with the vital sign sensors and configured to receive vital sign data from same for storage in the memory, the processor running an instance of the model for each casualty and providing the output to a dashboard; and a display in communication with the processor and configured to provide recommendations and/or vital sign data to an individual treating each casualty via the dashboard. In a further system embodiment, the model predicts vital signs of each casualty under a plurality of scenarios including the following four scenarios for sixty minutes after application of the tourniquet: providing the casualty no resuscitation fluid, providing the casualty one unit of resuscitation fluid in either the first 30 minutes or the second 30 minutes, and providing the casualty two units of resuscitation fluid with one unit in the first 30 minutes and the second unit in the second 30 minutes of the sixty minutes.

In a further system embodiment to the above system embodiments, the model is a recurrent neural network. In a further system embodiment to the above system embodiments, the model includes a plurality of layers between an input layer and an output layer, each layer having 128, 256, or 512 nodes.

In a further system embodiment to the above system embodiments, the model includes an input layer, a first feedforward layer, a GRU layer, a second feedforward layer, and an output layer where the outputs of the first feedforward layer is a n-dimensional feature vector that is inputted into the GRU layer, the outputs of the GRU layer is a m-dimensional feature vector that is inputted into the GRU layer and the second feedforward layer, the outputs of the second feedforward layer is a o-dimensional feature vector that is inputted into the output layer that provides the outputs for the model that are then inputs back into the model, and wherein n, m, and o are equal to the number of nodes of the respective layer. In a further system embodiment to the above system embodiments of the previous paragraphs, the model includes an input layer, a first feedforward layer, the first feedforward layer having a plurality of first feedforward layer nodes, where each first feed feedforward layer node receives the heart rate at a given time (“HR(t)”), the fluid infusion rate at said given time (“uₜ(t)”), and the systolic blood pressure at said given time (“SBP(t)”); a gated recurrent unit layer (“GRU”) having a plurality of GRU layer nodes for personalizing the prediction for the casualty based on a first dimensional feature vector produced by the first feedforward layer, and the GRU layer further receiving a second dimensional feature vector from the GRU layer previous time period on a second iteration of the model; a second feedforward layer having a plurality of second feedforward layer nodes, where each second feedforward layer node receives the second dimensional feature vector, the second feedforward layer outputs to an output layer that outputs a predicted value of the heart rate at a future time (“HR(t+1)”) and a predicted value of systolic blood pressure at a future time (“SBP(t+1)”), which then are provided as inputs back into the model, and wherein the model is repeatedly iterated for a predetermined time period, and the predetermined time period is sixty minutes.

In a further system embodiment to the above system embodiments, the model is trained on vital signs generated by a cardio-respiratory mathematical model to simulate future heart rates and systolic blood pressure based on inputted heart rates, systolic blood pressures, and infusion rates. In a further system embodiment to the above system embodiments, the model utilizes measured vital-sign data to update at least one hidden state to personalize the model for the casualty, and starting at application of hemorrhage control, the model continuously predicts the vital-sign data for each subsequent minute using the infusion rate of the associated scenario and feeding back into the model the calculated vital-signs until the model reaches a desired measuring time to allow for a comparison between the multiple scenarios.

### IV. BRIEF DESCRIPTION OF THE DRAWINGS

FIG. 1 illustrates a structure of the model according to at least one embodiment of the invention.

FIG. 2 illustrates events and time intervals used to create different scenarios representing an initial hemorrhage-inducing trauma, tourniquet application, and subsequent fluid resuscitation treatment.

FIG. 3 illustrates transfusion rates of four fluid treatment options according to at least one embodiment of the invention.

FIGS. 4A-4C illustrate a dashboard that may be useful in at least one embodiment according to the invention.

FIG. 5 illustrates an outline of the methodology used to develop and assess the AI method for personalized resource allocation of hemorrhage casualties.

FIG. 6 illustrates average validation errors s for different numbers (128, 256, and 512) of nodes used in the three layers of the model.

FIG. 7 illustrates a procedure for selection of CR model parameter sets representing individuals used to generate vital-sign trajectories associated with the simulated hemorrhage and treatment scenarios.

FIG. 8 illustrates the range of bleeding times and blood-volume losses used to create different hemorrhage scenarios.

FIG. 9 illustrates a distribution of heart rate (HR) and systolic blood pressure (SBP) for the cohort of 160 trauma casualties before (green circles) and after (red squares) hemorrhage with the range within the black dashed lines represents the healthy initial range.

FIGS. 10A and 10B illustrate a comparison of Vampire- and AI-based allocation methods for the single casualty in Analysis 1 with FIG. 10A showing heart rate (HR) and FIG. 10B showing systolic blood pressure (SBP) over time, where t₁ denotes the time for tourniquet application and t₂ represents the time for initiation of fluid resuscitation, where the horizontal black solid lines represent the boundaries of the healthy target range, the thick solid lines represent vital signs during the hemorrhage phase, the yellow dashed lines denote vital signs with no fluid transfusion, and the green dash-dotted lines represent vital signs after receiving 1 unit of fluids (e.g., approximately 450 milliliters of whole blood) at t₂ infused for 30 minutes during the treatment phase.

FIGS. 11A and 11B illustrate a comparison of fluid allocations based on the CR model, AI predictions, and the Vampire Program for different numbers of available fluid units with FIG. 11A showing the number of casualties restored to the healthy target range and FIG. 11B showing the excessive use of fluid units (number of fluid units used more than required based on the gold-standard CR results) where the shaded areas represent two standard errors of the mean.

FIGS. 12 and 13 are Tables 2 and 3 that were too large for reproduction in the text.

FIG. 14 illustrates classification results of the linear support vector machine algorithm for vital-sign trajectories at the end of fluid resuscitation at time t₃ for the scenarios 1) when tourniquet application at t₁ controlled bleeding and 2) when tourniquet application at t₁ did not control all bleeding because there was additional non-compressible bleeding.

### V. DETAILED DESCRIPTION

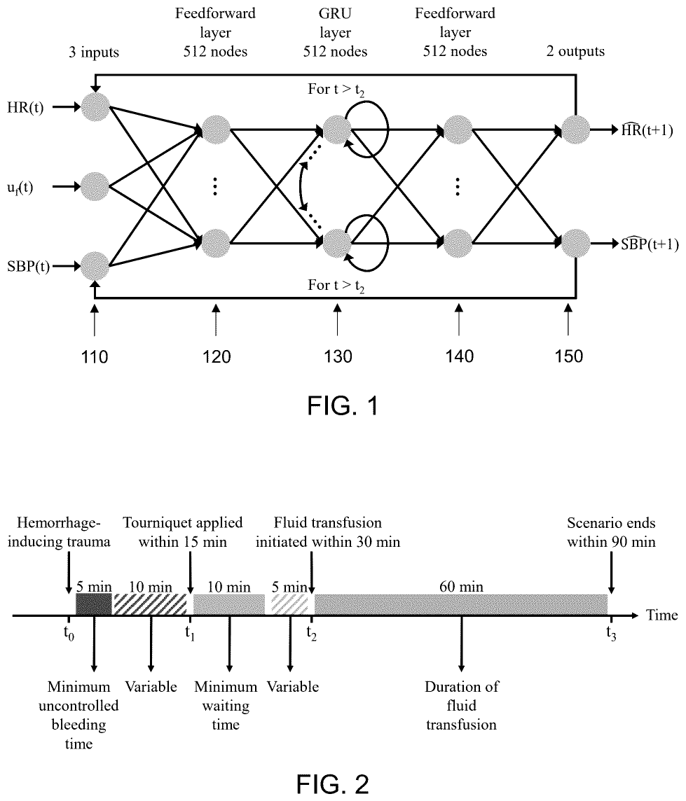

In at least one embodiment, as illustrated in FIG. 1, the model includes a neural network having multiple layers. In at least one embodiment, the layers include an input layer 110, a first feedforward layer 120, a gated recurrent (GRU) layer 130, a second feedforward layer 140, and an output layer 150 with the inputs being the heart rate (HR(t)), the systolic blood pressure (SBP(t)), and the infusion rate (uₜ(t)) and the output being heart rate ( [symbol] (t+1)) and systolic blood pressure ( [symbol] SBP(t+1)) at a future time.

Each node in the first feedforward layer 120 receives all three inputs from the input layer 110. The first feedforward layer 120 transforms the inputs into a n-dimensional feature vector where n is the number of nodes in the first feedforward layer 120.

The two inputs into the GRU layer 130 are the n-dimensional feature vector from the first feedforward layer 120 and the m-dimensional feature vector (hidden state) of the GRU layer 130 from the previous time step, where m is the number of nodes in the GRU layer 130. Because the GRU layer 130 uses the information from the previous time step, it is able to capture temporal dependencies and learn how features evolve over time. Unlike standard recurrent neural network models, the GRU layer 130 has gates to control how much of the previous hidden states to keep and forget, which generally leads to better predictive performance and more state training.

The input into the nodes of the second feedforward layer 140 receives the m-dimensional feature vector from the GRU layer 130. The second feedforward layer 140 transforms this vector into a representation that is suitable for the final output layer 150. The transformation enables the model to further refine its learned features before generating the prediction. The second feedforward layer 140 outputs its own o-dimensional feature vector to the output layer 150, which generates the two outputs, heart rate ( [symbol] (t+1)) and systolic blood pressure ( [symbol] (t+1)) at a future time, where o represents the number of nodes of the second feedforward layer 140. The two outputs from the output layer 150 are feed back into the model.

In at least one embodiment, n, m, and o are the same number. In at least one embodiment that number is 512. In an alternative embodiment, the mixture of feedforward and GRU layers is different and may include more than two feedforward layers and one GRU layer as long as the mix of nodes (e.g., 128, 256, or 512) considers the order of inputs as an aspect of its operation.

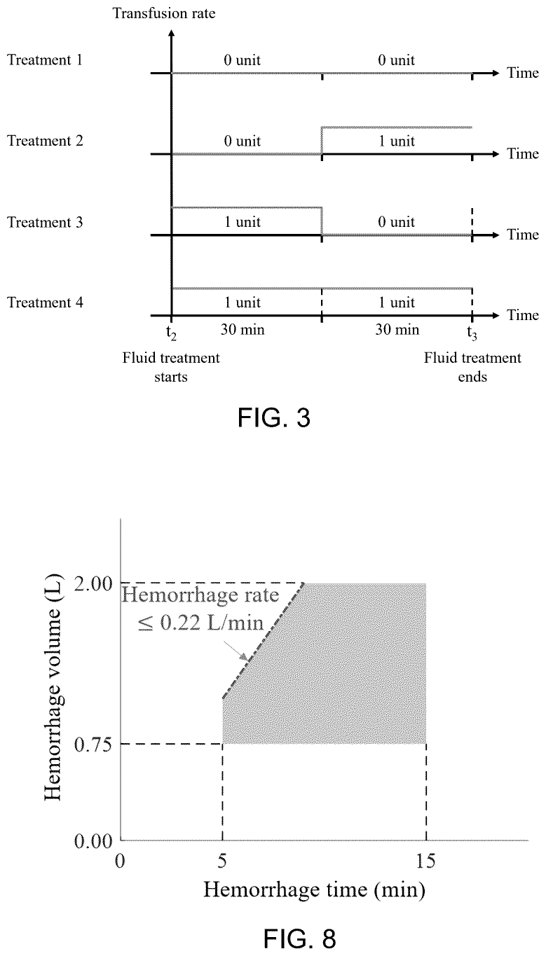

In at least one embodiment, the model uses eight to ten minutes of vital-sign data once the hemorrhage has been stopped, for example, after the application of a tourniquet in a limb. Although the amount of prior vital signs can vary depending on whether there are sufficient readings, for example, blood pressure readings may be more sporadic. At each time step t, the model receives three inputs: the fluid infusion rate [uₜ(t)], HR [HR(t)], and SBP [SBP(t)]. Consequently, it produces two outputs: the predicted HR [HR(t+1)] and SBP [SBP(t+1)] for the next time step (1 minute). To make personalized predictions, the model utilizes “measured” vital-sign dataform the preceding 8-10 minutes immediately before t₂ (i.e., from t₂−10 to t₂), as illustrated in FIG. 2, to update its hidden states. Starting at t₂, the GRU continuously predicts HR(t+1) and SBP(t+1) for each subsequent minute by using uₜ(t) and the fed back values of HR(t) and SBP(t) until the scenario is completed at t₃. Ultimately, the final predicted values HR and SBP at t₃ are used to evaluate the treatment outcomes, thus providing insight into the efficacy of the applied intervention at t₂. Given the number of minutes being simulated and the number of scenarios to be run, it is not possible for a human to perform the calculations in their head in the short period of time required to perform triage for a casualty while allowing the caregiver to move through any number of casualties in a short number of minutes. The model additionally is able to operate without the hinderance of stress, fatigue, lack of medical training, or potential environmental interruptions around the caregiver.

FIG. 2 illustrates events and time intervals used to create different scenarios representing an initial hemorrhage-inducing trauma, tourniquet application, and subsequent fluid resuscitation treatment. The injury at to is followed by a period of uncontrolled bleeding for a minimum of 5 minutes, after which a tourniquet is applied within a 10-minute interval, i.e., from 5-15 minutes after the injury. The tourniquet application at t₁ stops the bleeding, the fluid transfusion at t₂ is initiated at a time interval 10-15 minutes after t₁, and the transfusion continues for another 60 minutes until t₃. Different scenarios sample different time intervals between to and t₁ to apply a tourniquet and between t₁ and t₂ to start the transfusion, with blood transfusion starting between 15-30 minutes after the traumatic event. For this example, the maximum transfusion time is fixed at 60 minutes.

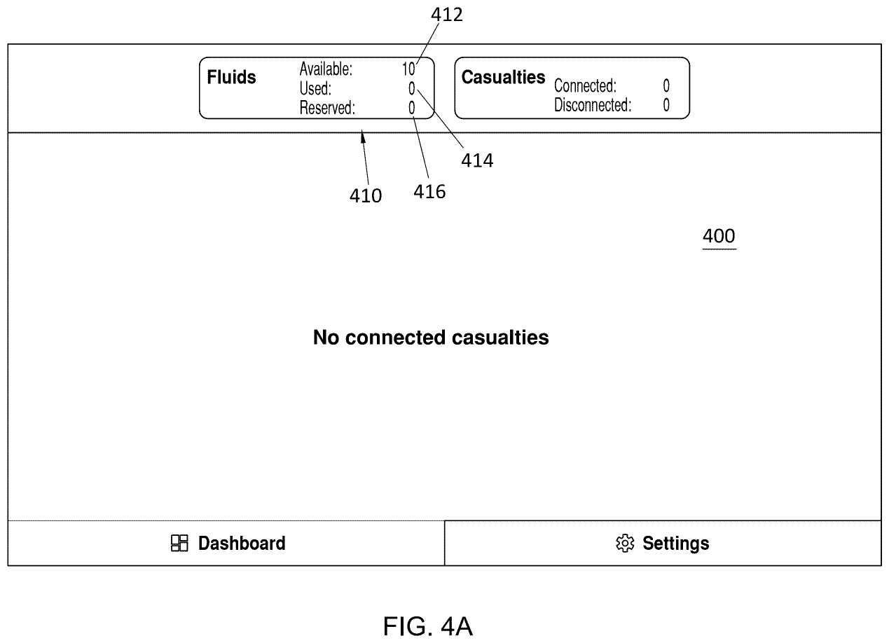

In a further embodiment, the model works through four scenarios: no infusion, an infusion in the first 30 minutes or the second 30 minutes after tourniquet application, and an infusion in both 30-minute periods as illustrated in FIG. 3. Depending on the outcome of the four scenarios, the scenario that uses the least infusion fluid while still returning the patient to healthy norms is used. Healthy norms are defined as being within the healthy target range of HR≤100 beats/minute and SBP≥100 mmHg. Although the model uses a generic resuscitation fluid, the model may instead or in addition use whole blood, blood products, saline, and crystalloids as available treatment options. In addition, the model may provide for smaller time windows and smaller infusion volumes, for example, 15-minute periods with half a resuscitation fluid unit with the time windows being at 15, 30, 45, and 60 minutes. Although the model was designed for the tourniquet stopping blood loss, the model may be adapted to consider non-compressible bleeding.

During operation, when the model reaches the end of the beginning or intermittent period window, the model performs another run using the recent vital-sign data to determine whether there is a change in treatment for the casualty.

It is worth noting that this approach, utilizing a recurrent artificial network model, is capable of predicting vital signs at any given future time, although prediction accuracy would decrease with an increasing prediction horizon. Nevertheless, modifying the method to account for the assessment of treatment outcomes at different time durations is a feasible option, allowing for a more detailed analysis of the effectiveness of fluid allocation strategies throughout the resuscitation process.

As discussed later, the theoretical benefits of the model include up to 46% additional casualties restored to healthy vital signs and up to a 119% efficiency increase in fluid utilization.

In at least one embodiment, the system includes vital-sign sensors for at least heart rate and blood pressure that are in communication with a processor on which the model resides that is in communication with a display (e.g., showing a dashboard) to provide information to at least one individual. The system further including a memory to store the vital-sign information, for example, the ten minutes or so of vital-sign information prior to application of a tourniquet or other hemorrhage control (e.g., a dressing or fibrinogen). In an alternative embodiment, the individual logs one or more vital signs into the device in which the processor is resident. In a further embodiment, the system may be resident on a wrist mounted device.

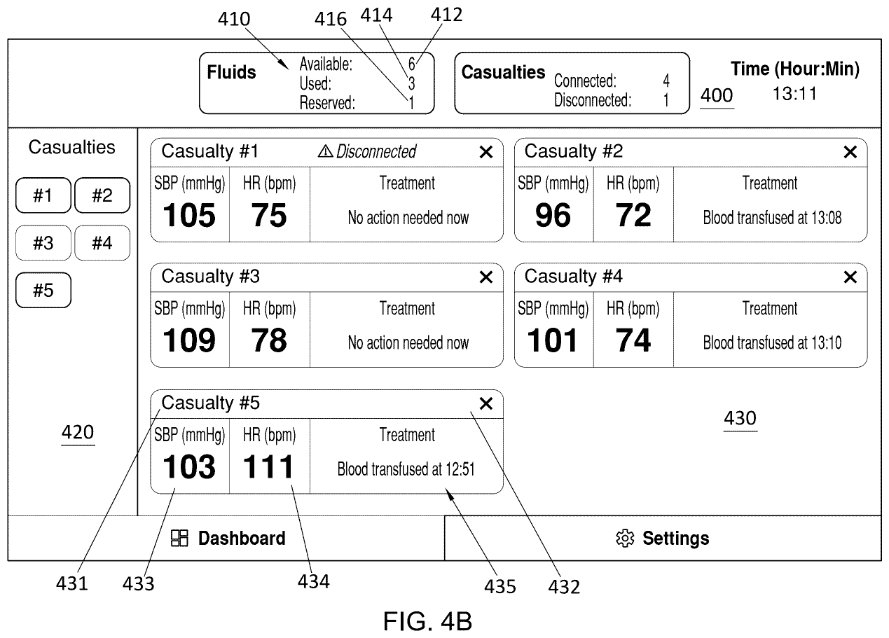

In at least one embodiment, the above-described model embodiments are included within a system that allows each casualty to be assigned a model instance and a dashboard for monitoring the different casualties including their vital signs and treatment options while maintaining an inventory of available resuscitation fluid units. FIGS. 4A-4C illustrate an example of the dashboard. FIG. 4A illustrates when the dashboard 400 is first brought up before connecting with any casualties, while FIG. 4B illustrates the dashboard previously or currently connected to five casualties. The dashboard 400 provides a running total 410 of resuscitation fluid units available 412, the number or units that have or are being administrated 414, and the number of units being held in reserve 416 based on casualty rates and expected casualties.

FIG. 4B illustrates an example of the system in use during a simulation with a pair of casualty windows: one window 420 with icons present that are color coded (e.g., #1, #2, #4, #4, and #5) and a second window 430 with vital sign information that is color coded with treatment status. The first window may use the following color-coding based on whether the casualty is disconnected (gray), connected and not requiring an infusion (dark navy color), and connected and requiring treatment (orange or red). The second window 430 identifies the casualty 431, whether they are connected or not 432, a blood pressure reading 433, a heart rate 434, and a treatment 435 with an optional time code. A similar color-coding of the first window 420 may be used in the second window 430 with the addition that a vital sign that is outside of healthy ranges is color coded (e.g., Casualty #2-SBP and Casualty #5-HR), for example in orange or red. Based on this disclosure, a person having ordinary skill in the art should appreciate different color-coding schemes could be used. In at least one embodiment, the icon in the first window 420 and/or the casualty title in the second window 430 can bring up additional information about that casualty.

FIG. 4C illustrates how, in at least one embodiment, the user is able to view additional information regarding the selected casualty 440. The additional information may include a graphical representation of historical to current vital signs 442 for the casualty with heart rate 4422 being largely continually monitored while blood pressure readings 4424 are intermittent as represented by the dots. This window 440 may also provide additional information regarding the treatment 444, which in the illustrated example the casualty did not receive a resuscitation fluid unit during the first 30-minute period, but did receive one in the second 30-minute period. The user is able to inform (or confirm) the system that a resuscitation fluid unit has been administered to the casualty. Additional details regarding the casualty are provided in a textual/numerical window 446 and includes connection status 4462, total time connected 4464, Bluetooth connection status 4466, current SBP 4468, and current HR 4469.

The system is able to allocate available resuscitation fluid based upon demand and expected demand based on the number of instances running and the level of resuscitation fluid units required to balance the use of the existing supply of resuscitation fluid. For example, if a large percentage of a military unit has been wounded and there is minimal risk for additional casualties, then the system will be more aggressive in the administration of resuscitation fluid including providing more than one resuscitation fluid unit by in part holding fewer resuscitation fluid units in reserve. Conversely, if there is a risk for more casualties, then the system will be more conservative by providing recommendations to conserve the use of resuscitation fluid by avoiding treatments that require multiple resuscitation fluid units and holding a larger percentage of resuscitation fluid units in reserve. For a fixed number of casualties requiring fluid and a fixed number of available units of fluid, the system will first identify those casualties who require 1 unit and those who require 2 units. If the number of available units of fluid is not sufficient to treat every casualty, the system will first treat casualties who require 1 unit only. In some cases, it would infuse the 1 unit at the current time and in other cases it would “reserve” the 1-unit fluid for a later infusion time. In either case, the fluid infusion is expected to restore the casualty's vital signs to healthy norms at 60 minutes. If after treating every casualty who requires 1 unit of fluid there is still some units left, but not enough to treat every casualty who requires 2 units, the system will recommend giving 1 unit at the current time for a casualty and “reserve” 1 unit for this casualty for infusion at a later time. In this way, the casualty will receive a total of 2 units with the expectation that the two infusions will restore the casualty's vital signs to healthy norms at 60 minutes. In this case, other casualties requiring 2 units may not be treated, if fluid is not available. In at least one embodiment when supplies are limited, the earliest arriving casualty that requires two units of resuscitation fluid will receive resuscitation fluid over a later arriving casualty that requires two units of resuscitation fluid. Similarly, if the supplies diminish to a point where there are insufficient resuscitation fluid units available for the casualties requiring one unit, then the earlier arriving casualty will be given priority. An alternative approach where these two casualties receive 1 unit each, when each casualty actually requires 2 units each to be restored, would not be optimal because it would not restore neither casualty.

In at least one embodiment, the system is able to handle an uneven flow of casualties particularly when casualties are arriving and/or being triaged at different times by running individual model instances that provide recommendations for that particular casualty while tracking the respective different times and automatically processing vital sign data at the end of the time period.

In at least one embodiment, the vital signs are sporadic and/or have noise in them resulting in less-than-ideal data for inputting into the model. The SBP measurements were adjusted to be spaced out from each other and not continuous (e.g., 3, 4, 5, 2.5-5, or 2.5-4 minute intervals). In a further embodiment, the model will smooth or repeat the SBP measurements between the measurement points to provide a continuous flow of SBP measurements along with heart rate measurements. In at least one embodiment, the noise that might be present is addressed by smoothing or avoiding outlier measurements from the remaining measurements.

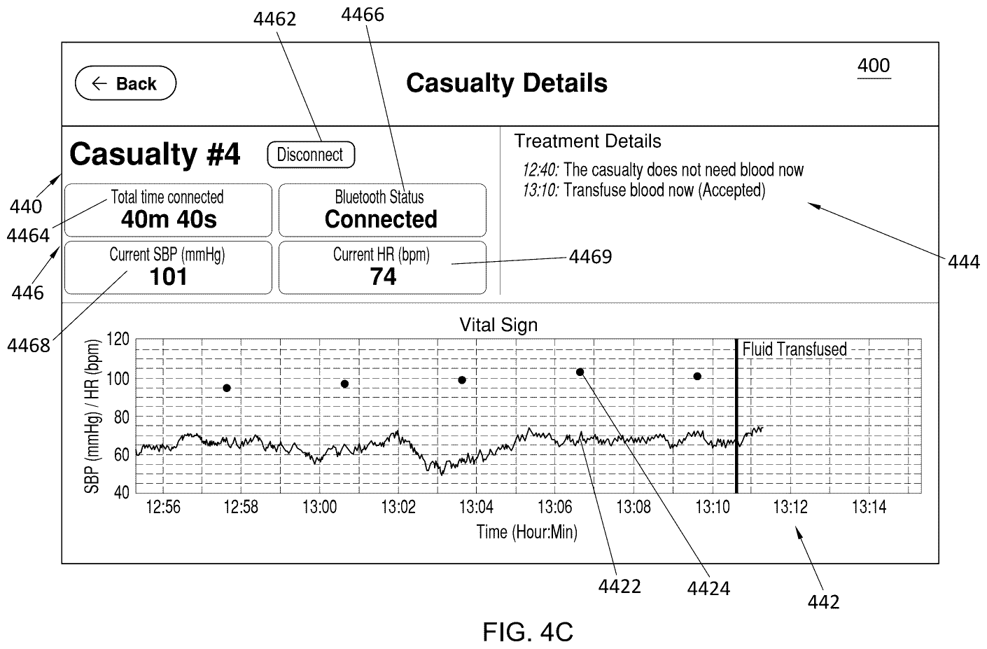

FIG. 5 provides an overview of the AI model training and the evaluation process where the model was compared to the Vampire Program. I) Synthetic-data generation: Use the CR model to perform simulations and generate synthetic trauma casualties with associated vital-sign trajectories, for a given hemorrhage-inducing trauma condition and each of four fluid treatment options. II) AI-model development: Use the CR-generated synthetic data, perform a 5-fold nested cross-validation to develop AI models that use 10 minutes of pre-fluid-treatment vital signs to predict personalized vital signs 60 minutes into the future after fluid treatment. Ill) AI and Vampire assessment: Use the CR-generated vital-sign data to compare the outcomes in terms of the number of restored casualties to a “safe” physiological state (i.e., healthy norms) and the amount of fluid utilization for the optimal fluid treatments allocated by the AI model and the Vampire Program as well as the CR-based optimal fluid treatment. The CR model was used to generate thousands of vital-signs sets to train the model and then to test the model. The training sets included the preceding ten minutes of vital signs followed by sixty minutes of vital signs.

The CR model was used to generate synthetic data that capture the time-dependent evolution of vital signs associated with hemorrhage and subsequent fluid resuscitation treatments. The CR model integrates cardiovascular and respiratory processes with their regulatory mechanisms to provide physiologically appropriate vital-sign time-course data that mimic the human response to hemorrhage and related treatments. The model consists of 74 ordinary differential and algebraic equations with 74 parameters. The inputs to the CR model include the rate of hemorrhage, rate of fluid resuscitation, minute ventilation, and fraction of inspired oxygen; the model outputs consist of arterial blood pressure [systolic (SBP), diastolic, and mean], heart rate (HR), partial pressure of end-tidal carbon dioxide, and oxygen saturation.

The CR model utilizes a lumped-parameter formulation based on first principles (conservation of mass) to represent fluid balances within vascular compartments and gas balances within the lungs and tissues, as well as a compartmental phenomenological formulation to represent the regulatory mechanisms and couplings between the cardiovascular and respiratory modules. Through this framework, the CR model enables the simulation of hemorrhage, fluid resuscitation, and respiratory perturbations, facilitating the generation of synthetic data that simulate injury and treatment scenarios of interest. It is important to note that a current limitation of the CR model includes the inability to account for specific types of resuscitation fluids, as the CR model solely considers the volume of fluid administered.

FIG. 5 illustrates a 5-fold nested cross-validation approach was used to develop and test the AI model predictions through the following process consisting of four steps using N_{F} simulated casualties generated by the CR model.

First, the cohort of N_{F} simulated casualties was divided into five groups of N_{F}/5 casualties each. Given that the initial vital signs are associated with subsequent responses to fluid perturbations, a balanced set of initial vital signs in each group was selected to avoid biasing the model. Thus, the healthy vital-sign target range, defined as heart rates (HRs) between 60-100 beats/minute and systolic blood pressures (SBPs) between 100-140 mmHg, was divided into four equally spaced regions and balanced each group to include an equal number of individuals in each quadrant.

Next, for the outer loop of the cross-validation, one group was treated as the outer test set and merged the other four groups to form the outer training set. This process was repeated iteratively for all five groups, ensuring that each group served as the test set once.

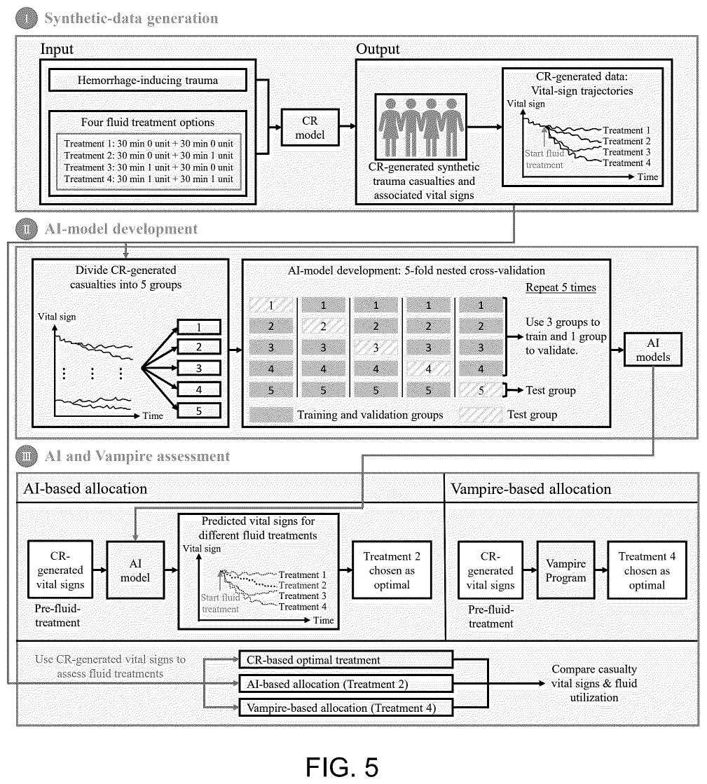

Then, within the outer training set, an inner loop was established. This involved training the model on three of the four groups within the outer training set and validating it on the remaining group. This training and validation process was repeated for all four possible combinations of groups. To optimize the weights of the AI model, the Adam optimization algorithm (Jais et al., 2019) was employed and aimed to minimize the sum of the normalized prediction errors s of the vital signs (HR and SBP) over the 60-minute duration of fluid resuscitation, as defined by Equation (1) below:

$$ε = {\sum }_{t = 1}^{60} \{ {[ [ ( t ) - HR ( t ) ] / 150 ]}^{2} + {[ [ ( t ) - S B P ( t ) ] / 110 ]}^{2} \} / 60 \qquad ( 1 )$$

where t denotes a time index; HR(t) and SBP(t) denote “measured” vital signs generated by the CR model; [symbol] (t) and [symbol] (t) represent the AI-model-predicted HR and SBP at time t, respectively; and 150 and 110 represent normalization factors indicative of the ranges observed during the CR-model simulations. Notably, through experimentation, it was observed that s was predominantly influenced by the number of nodes in the GRU layer. Consequently, the same number of nodes were used across the three layers and tested different numbers of nodes (e.g., 128, 256, and 512), as plotted in FIG. 6. The five types of markers (blue triangle, green square, purple diamond, red star, and black circle) represent the average validation error s for the five different groups of simulated casualties. To avoid over-parameterization, a larger number of nodes were not considered as they would have led to an excessive number of model parameters. For other hyperparameters, a 25% dropout rate was set for all three hidden layers, utilized a default learning rate of 0.001, and stopped the training process when the validation error did not improve over 2,000 epochs. The optimal hyperparameters were selected as those that yielded the lowest average validation error s over the four inner models. The model with 512 nodes in each layer consistently exhibited the lowest average E, leading to its selection in the model.

Finally, to assess the prediction accuracy of the model, an ensemble model was created by averaging the predictions obtained from the four inner models with the best hyperparameters. This ensemble model was tested on the corresponding outer test set. This process was repeated for all five groups, yielding five distinct test errors s that allowed evaluation of the model's performance across different test sets. Moreover, to quantitatively evaluate the model's performance in capturing the dynamics of HR and SBP during the fluid resuscitation process, the root mean square error was computed between the AI-model predictions and the synthetic data generated by the CR model for HR (δₕ) and SBP (δₛ) over 60 minutes of fluid transfusion, as defined by Equations (2a) and (2b) below:

$${δ}_{h} = {{\sum }_{t = 1}^{60} [ ( t ) - H R ( t ) ]}^{2} / 60 \qquad ( 2 a )$$

$${δ}_{s} = {{\sum }_{t = 1}^{60} [ ( t ) - SBP ( t ) ]}^{2} / 60 \qquad ( 2 b )$$

The model was used to optimize fluid allocation and its performance was evaluated by comparing it with the Vampire Program, a DoD guideline used to guide fluid resuscitation based on HR, SBP, and the presence of amputation to provide an evaluation of the model. However, because the CR model only predicts vital signs, the analysis focused solely on HR and SBP. For the sake of simplicity, the Vampire Program was modified into a two-step process for the study: 1) prior to fluid resuscitation, if the vital signs of the casualty were not within the healthy target range [HR≤100 beats/minute and SBP≥100 mmHg], a transfusion with 1 unit of fluid for 30 minutes was initiated and 2) after the initial 30 minutes, an additional unit of fluid was administered if the CR-model-simulated vital signs continued to fall outside of the healthy target range.

Similarly, the developed fluid allocation strategy also consisted of a two-step process: 1) before initiating fluid resuscitation, the model trained on 10 minutes of data to predict the personalized outcome of the casualty at 60 minutes for each of the four treatment options was employed and the one that used the least amount of fluids to restore the casualty's vital signs to the healthy target range was selected. Then, the model used the CR-generated data to obtain the outcome of the selected transfusion for the initial 30 minutes and 2) after the 30 minutes, the model used the available 40 minutes (10+30 minutes) of CR-model-simulated vital signs to update the model and predict the outcome at 60 minutes for each of two treatment options (0 or 1 unit for the final 30 minutes). Similarly, the treatment that used the least number of resuscitation fluid units to restore the casualty was selected. When allocating fluids for a casualty within one of the five groups of N_{F}/5 casualties, the model trained on the other four groups was used to predict the casualty's vital signs. As a result, the models employed in the study do not possess any prior information regarding the casualties they are treating, ensuring a fair and unbiased allocation process. Moreover, to achieve the maximum number of casualties restored to the healthy target range with the given available fluid units, an optimal allocation strategy should refrain from administering fluids to casualties who do not require them or who cannot be restored to the healthy target range even with 2 units. Instead, the method should prioritize administering fluids to casualties in need of 1 unit, followed by those in need of 2 units.

To perform a side-by-side comparison between the AI- and Vampire-based allocation methods, the evaluation conducted three different analyses. Analysis 1 served as a simple demonstration of the advantages offered by the AI allocation method, while the subsequent two analyses provided deeper insights into the relative performance and effectiveness of the two allocation methods under diverse scenarios.

Analysis 1. The evaluation employed the two methods to allocate fluids to one casualty and compared the number of used fluid units to restore the casualty to the healthy vital-sign target range.

Analysis 2. The evaluation expanded the evaluation by allocating varying units of fluids to N_{F}/5 casualties within each group, employing both allocation methods. The first comparison was the number of casualties restored to the healthy target range for each of the two allocation methods as well as the CR-based allocation method, which provided an upper bound of the maximum number of possible restored casualties. Regarding the CR-based allocation method, the CR-generated data was used to obtain the outcomes at 60 minutes for all four treatments and selected the one that used the least number of resuscitation fluid units to restore the casualty's vital signs to the healthy target range. Similar to the AI-based allocation method, this method also prioritized administering fluids to casualties in need of 1 unit, followed by those in need of 2 units. Next, a comparison was made between the excessive use of fluids in the AI- and Vampire-based allocation methods (the number of fluid units used more than required based on the CR model).

Analysis 3. The evaluation explored the performance of the two allocation methods in a scenario involving the allocation of different units of fluids to varying numbers of casualties. To achieve this, the N_{F}/5 casualties of each group were divided into different group configurations, including two groups of N_{F}/10 casualties, four groups of N_{F}/20 casualties, and eight groups of N_{F}/40 casualties. Subsequently, both the AI- and Vampire-based allocation methods were utilized to distribute fluids to each group. The study specifically examined the fraction of casualties restored to vital signs within the healthy target range using the AI-based method compared to the Vampire-based method. Additionally, the relative ratio R of fluid-utilization efficiencies were computed between the two methods, as defined by Equation (3) below:

$$R = ( {N}_{A} / {U}_{A} ) / ( {N}_{V} / {U}_{V} ) \qquad ( 3 )$$

where N_{A} and N_{V} denote the total number of casualties restored to the healthy target range by the AI- and Vampire-based allocations, respectively, and U_{A} and U_{V} represent the total number of units of fluid utilized by the two methods. Hence, R>1.00 indicates a greater efficiency of the AI method over the Vampire Program allocation. To prevent an “undefined” ratio R, we only evaluated R when at least 1 unit of fluid was used (i.e., U_{A} and U_{V}≠0 units).

In the analyses above, the assumption was that the tourniquet applied at time t₁ set the bleeding rate to zero (completely stopped all bleeding) and that there was no non-compressible bleeding present. However, the AI model was capable to detect cases where the casualties experienced non-compressible bleeding. As hemorrhage typically leads to an increase in HR and a decrease in SBP, casualties with non-compressible bleeding are more likely to exhibit higher HR and lower SBP values. As the AI model does not account for non-compressible bleeding, the measured vital signs may deviate from the predicted values if bleeding persists. Therefore, by assessing the disparity between the measured (as predicted by the CR model, in the comparison between approaches) and the model-predicted vital signs, it becomes possible to identify whether a casualty is still experiencing non-compressible bleeding or not.

To verify this capability, aside from the previously generated 4N_{F} trajectories referred to as the controlled bleeding scenario, the CR model generated an additional set of simulations for the cohort of N_{F} casualties. The simulations were conducted using the same bleeding rate but varied the fractions of non-compressible bleeding to 10%, 20%, 30%, 40%, and 50% of the total bleeding rate. Subsequently, all four treatment options were applied to these simulated trajectories, resulting in a total of N_{N} completed trajectories of non-compressible bleeding.

To classify the controlled and non-compressible bleeding scenarios, a support vector machine (SVM) with a linear kernel (Burges, 1998) was utilized. Given the discrepancy in the number of trajectories between the two scenarios (4N_{F} trajectories for controlled bleeding and N_{N} trajectories for non-compressible bleeding), the trajectories were weighted inversely proportional to their respective numbers for classification, ensuring that trajectories from both scenarios contributed equally to the classification analysis. After implementing the SVM algorithm on the two scenarios, the classification accuracy was computed of each scenario to assess the performance of the detection method.

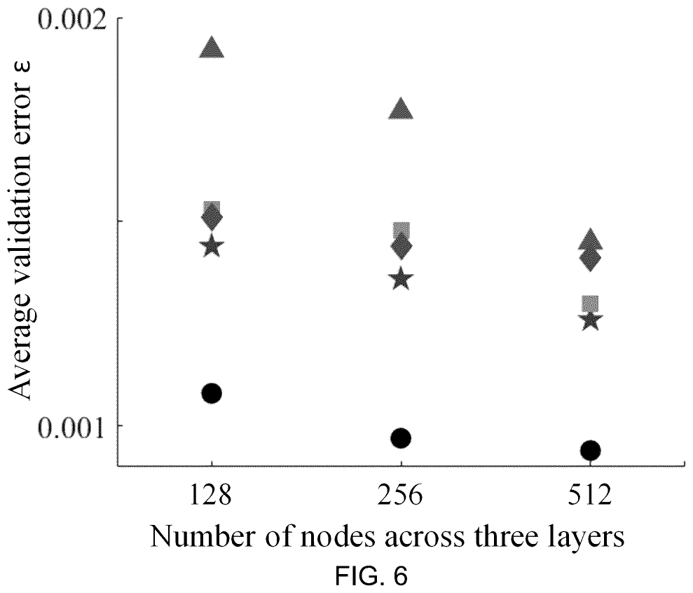

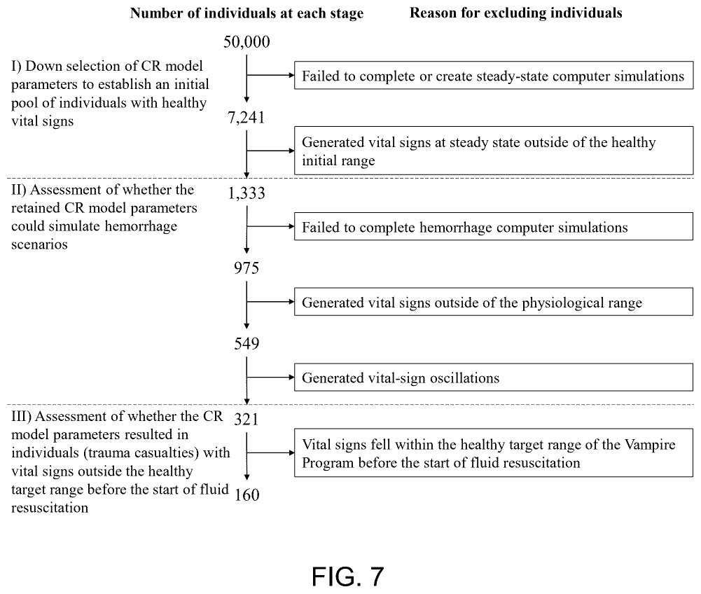

As illustrated in FIG. 7, the selection procedure includes three different stages (1-111) in order to generate a broad range of individuals with vital signs in the healthy target range before hemorrhage and outside of this range after hemorrhage onset, to successfully simulate different degrees of moderate to severe hemorrhage. A pool of 50,000 individuals was down-selected to a pool of 160 individuals (trauma casualties) with vital signs outside of the healthy target range of the Vampire Program (HR≤100 beats/minute and SBP≥100 mmHg) before the start of fluid resuscitation (t₂), through three stages. FIG. 7 outlines the down-selection process. In particular, Stage 1) established an initial pool of 1,333 individuals with healthy vital signs; Stage 11) retained 321 individuals who completed the hemorrhage scenarios; and Stage Ill) assigned one random bleeding scenario within the shaded region in FIG. 8 to each of the remaining 321 individuals and excluded 160 individuals whose vital signs fell within the healthy target range of the Vampire Program at t₂. The pentagon-shaded area in FIG. 8 defines the range of bleeding parameters used to create Class II and Ill hemorrhage cases for this evaluation, compatible with bleeding times of 5-15 minutes and blood-volume losses of 0.75-2.00 L. The dash-dotted edge of the pentagon represents the 0.22 L/minute maximum rate of hemorrhage for these scenarios. Finally, the evaluation randomly deselected one individual to generate a final cohort of N_{F}=160 trauma casualties who could be evenly divided into five groups for data generation.

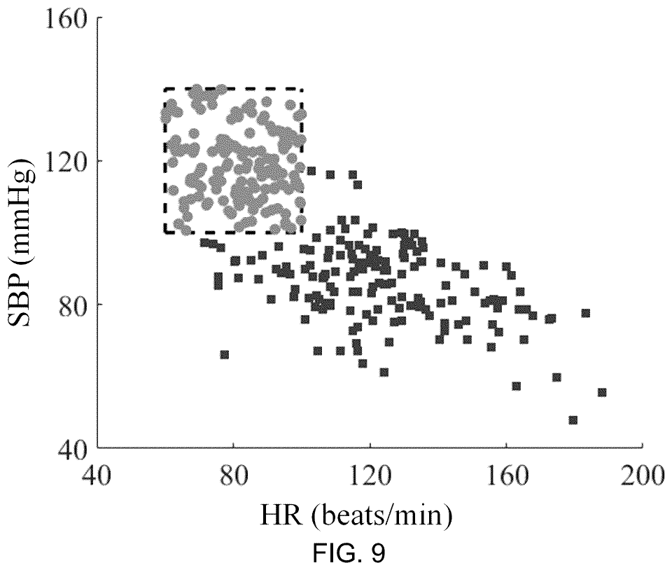

To evaluate the range and variation of vital signs for the development of the AI model, the evaluation examined the distribution of their values before and after hemorrhage among the generated trauma casualties. FIG. 9 shows the HR and SBP values for each of the 160 casualties at the beginning of the injury scenario (to) in FIG. 2, representing initial vital signs (circles largely within the dashed box) in the healthy initial range and after hemorrhage at t₁ (red squares). The initial vital signs were distributed across the entire healthy initial range, ensuring that the generated population captured a broad range of healthy baseline vital signs. In addition, the simulated hemorrhage scenarios led to an elevation in HR accompanied by a decrease in SBP, with HR values ranging between 70-100 beats/minute and SBP values between 40-120 mmHg (FIG. 9, squares). The upper bound of HR and the lower bound of SBP spanned the range of their respective physiological limits, reflecting the ability of the injury hemorrhage scenarios to induce significant changes in vital signs. Thus, the generated data captured a high degree of individual variability, with a range of vital-sign changes that provided a diverse set of synthetic data for the development of the model.

The evaluation divided the cohort of N_{F}=160 trauma casualties into five groups of 32 (N_{F}/5) casualties each and performed a 5-fold nested cross-validation. The evaluation first examined the three hidden layers of the recurrent neural network using 128, 256, and 512 nodes each and selected 512 nodes, as this consistently yielded the lowest average validation error s between the model predictions and the synthetic data over 60 minutes of fluid transfusion. For results of the 128- and 256-node models, see FIG. 6.

Table 1 below shows the average and standard deviation (SD) of the RMSEs between the AI-model predictions and the synthetic data for HR and SBP. As the evaluation used the 5-fold nested cross-validation method, the inventors trained and validated 20 (5×4) AI models. The average training RMSEs of HR (δₕ) and SBP (δₛ) over the 20 models were 3.4 (SD=0.9) beats/minute for HR and 2.5 (SD=0.7) mmHg for SBP. Likewise, the average validation δₕ and δₛ were 4.2 (SD=1.0) beats/minute for HR and 2.8 (SD=0.5) mmHg for SBP. This correspondence between the training and validation errors indicated that the models were not over-fitted to the training data and generalized well to unseen validation data.

TABLE 1
Training, validation, and test root mean square error (RMSE) between
the Al-model predictions and the synthetic data generated by
the cardio-respiratory model over 60 minutes of fluid transfusion
for heart rate (HR) and systolic blood pressure (SBP).

| HR RMSE (δₕ) | | | SBP RMSE (δₛ) | | |
|---|---|---|---|---|---|
| (beats/minute) | | | (mmHg) | | |
| Training | Validation | Test | Training | Validation | Test |
| (N = 20) | (N = 20) | (N = 5) | (N = 20) | (N = 20) | (N = 5) |
| 3.4 (0.9) | 4.2 (1.0) | 4.3 (0.7) | 2.5 (0.7) | 2.8 (0.5) | 2.9 (0.5) |

Data are presented as mean (standard deviation). N represents the number of AI models.

Finally, the average test δₕ and bs over the five groups were 4.3 (SD=0.7) beats/minute for HR and 2.9 (SD=0.5) mmHg for SBP. Although these errors were larger than the validation error, the absolute errors were comparable to the level of vital-sign monitor instrumental accuracy, indicating that the AI model captured changes in HR and SBP associated with fluid resuscitation treatment of a broad range of hemorrhage scenarios in a population of diverse casualties.

Three analyses were conducted to evaluate the effectiveness of fluid allocations based on the AI predictions and the Vampire Program. Given that the CR model provides the ground truth for changes in vital signs upon hemorrhage as well as treatment, the evaluation compared both allocation methods to the CR model and assessed the relative performance of each method.

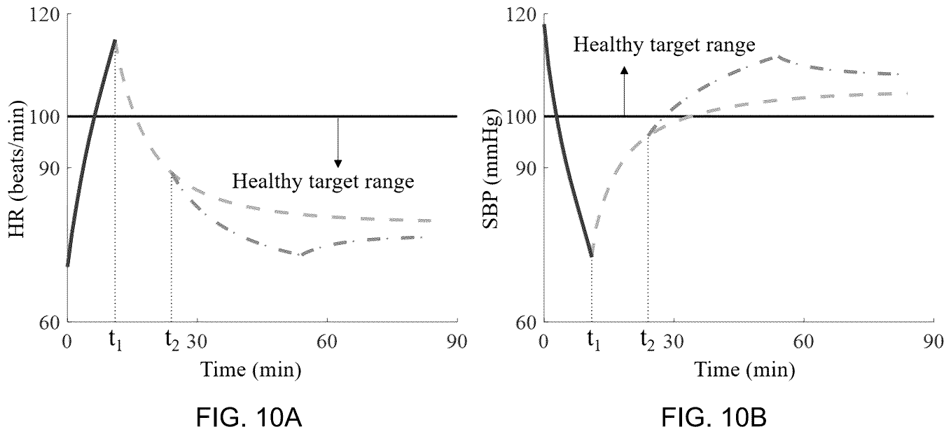

In Analysis 1, the evaluation examined fluid allocation methods using one casualty, which like all simulated casualties had vital signs at time t₂ outside of the healthy target range of the Vampire Program (FIGS. 10A and 10B). This case was selected to highlight one possible advantage of the AI-based method, where the best fluid allocation to restore the casualty to the healthy target range according to the CR model was by giving the casualty 0 units of fluid because tourniquet application alone at time t₁ was sufficient. FIGS. 10A-10B show that at t₂ the casualty's HR fell inside the healthy target range (FIG. 10A) while the SBP did not (FIG. 10B). Consequently, the Vampire Program guideline initially recommended transfusing 1 unit of resuscitation fluid for the initial 30-minute period. Following this period, the vital signs returned to the healthy target range (FIGS. 10A-10B) and the guideline recommended discontinuing the resuscitation. In contrast, the allocation choice using the model was based on initially predicting the outcomes of all four treatment options for the casualty. Thus, the AI model correctly predicted that all four treatment options would result in outcomes within the healthy target range and, hence, no fluid was required for this casualty.

The ability of the model to choose the optimal allocation strategy at the outset and ignore fixed vital-sign guidelines for fluid resuscitation allowed us to correctly transfuse 0 units of fluid to the casualty and return it to a healthy vital-sign state, while saving fluids. The predicted upfront knowledge of treatment outcomes provided the AI-based allocation a clear advantage in this case. However, the AI-based allocation method does not always outperform the Vampire Program because the model-predicted vital signs have small errors when compared to the synthetic data generated by the CR model.

In Analysis 2, the evaluation used a fixed number of casualties (N_{F}/5=32) and introduced a varying number of available fluid units (0-42) for resuscitation. The evaluation compared 1) the total number of casualties restored to the healthy target range by the two allocation methods compared to the CR model and 2) the excessive recommendation and use of fluids generated by the allocation methods. To make a statistical comparison, the evaluation used the average results derived from the five groups, each made up of 32 casualties.

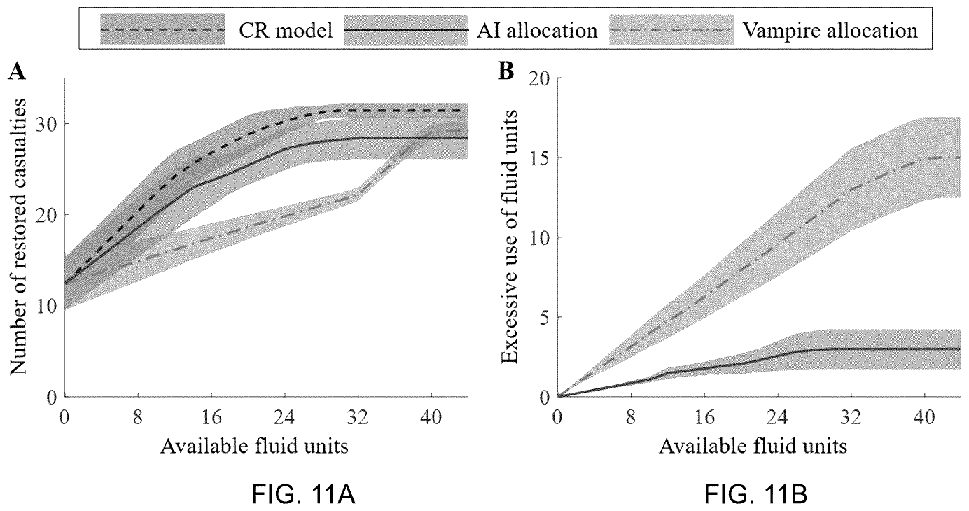

FIG. 11A shows the number of restored casualties for the allocation methods based on the CR model (dashed line and corresponding shaded area), AI predictions (solid line and corresponding shaded area), and the Vampire Program (dash-dotted line and corresponding shaded area). The lines and shaded areas represent the mean and two standard errors (SEs) of the mean. On average across the five groups, 12.4 casualties (2 SE=2.9) were restored to the healthy target range without any fluid administration. As the number of available fluid units for resuscitation increased, the average number of restored casualties rose. For the CR model (the gold standard), the average number increased at a rate of 1.0 casualty/unit until 10 units were administered, resulting in 22.4 (2 SE=2.9) casualties restored to the healthy target range. Subsequently, the rate decreased to 0.7 casualties/unit until 16 units, leading to an average of 26.8 (2 SE=2.1) restored casualties. Further increments in fluid units resulted in a decline in the rate to 0.5 casualties/unit until 20 units, with an average of 28.8 (2 SE=1.9) restored casualties. Beyond 20 units, the rate continued to decrease, eventually stabilizing at 31.4 (2 SE=0.8) restored casualties with 30 units. In contrast, the AI-based allocation restored fewer casualties compared to the CR baseline for any given number of fluid units. This was attributed to errors in the AI-model predictions and the resulting sub-optimal fluid allocation compared to the CR model. The average number of casualties restored increased at a rate of 0.8 casualties/unit until 14 units, resulting in 23.0 (2 SE=3.4) restored casualties. Then, the rate decreased to 0.4 casualties/unit until 24 units, with an average of 27.2 (2 SE=2.3) restored casualties. At the saturation point of 32 units, the average number of restored casualties stabilized at 28.4 (2 SE=2.3). Comparatively, the Vampire-based allocation restored fewer casualties than the AI-based allocation. The difference was consistently larger in resource-limited conditions where the number of units was below 32. The average number of restored casualties increased only at a rate of 0.3 casualties/unit until 32 units, resulting in 22.2 (2 SE=0.7) casualties in the healthy target range. Subsequently, the rate sharply increased to 0.9 casualties/unit until 40 units, yielding an average of 29.0 (2 SE=0.9) restored casualties. Beyond this point, the rate became negligible, and the average number of restored casualties stabilized at 29.2 (2 SE=1.0) up to the maximum of 42 units used for resuscitation.

FIG. 11B shows the excessive use of fluids for the two allocation methods compared to the true minimum number based on the CR model. The excessive use of fluid units increased as the number of available fluid units increased, i.e., the inefficiency increased with increasing availability of fluids. For the AI-based allocation, the average excessive use of fluid units increased roughly at a rate of 0.1 per available fluid units. It peaked at 3.0 (2 SE=1.3) units with 30 units of available fluid and saturated at this level. Comparatively, the Vampire-based allocation exhibited a much larger excessive use of fluid units, which increased at a rate of 0.4 per available fluid units until 32 units, resulting in an average of 13.0 (2 SE=2.6) units of excessive fluid. Upon reaching 42 units, the excessive use reached a plateau, stabilizing at an average of 15.0 (2 SE=2.5) units.

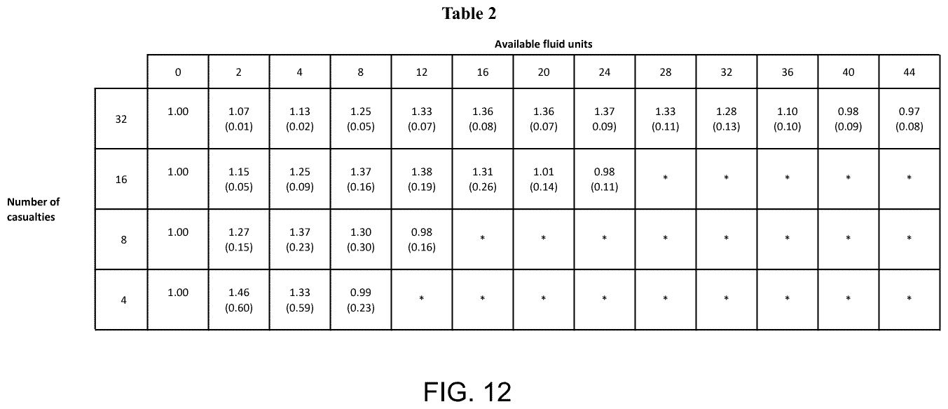

In Analysis 3, the evaluation examined variations both in the number of available fluid units (0-42) and the number of casualties (4, 8, 16, and 32) potentially requiring fluid resuscitation. Table 2 (FIG. 12) shows the fraction of casualties restored to the healthy target range using the AI-based allocation method compared to the Vampire-based allocation. The results consistently demonstrated that the AI-based method was more efficient (fraction>1.00) in resource-limited conditions across different numbers of casualties. In the case of 32 casualties, corresponding to the data shown in FIG. 11A, the fraction of casualties restored to the healthy target range increased steadily until reaching 24 units of available fluid. At this point, the fraction peaked at 1.37 (SD=0.09), indicating that the AI-based allocation method restored 37% more casualties than the Vampire-based allocation. However, beyond this point, the fraction of restored casualties started to decrease, and the two allocation methods became comparable when a larger number of fluid units were available, resulting in a fraction of ˜1.00 after 40 fluid units.

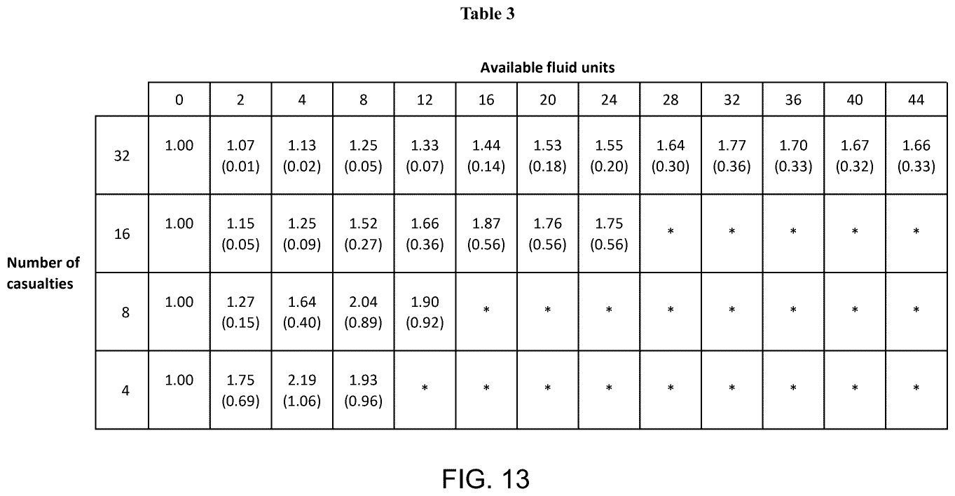

To gauge fluid-utilization efficiency, the relative efficiency R in Equation (2) (i.e., the number of casualties restored per utilized fluid unit) between the two methods was compared. R values above 1.00 indicate that, on average, the AI-based method was more efficient than the Vampire-based method. Table 3 (FIG. 13) shows this fluid-utilization efficiency metric for variable numbers of available fluid units and hemorrhage cases potentially requiring fluid resuscitation. An observed result was that R ranged from 1.07 (SD=0.01) to 2.19 (SD=1.06), demonstrating a consistently improved fluid-utilization efficiency of the AI-based allocation method over the Vampire-based allocation. Similar to as in Table 2, the relative efficiency exhibited an increasing trend followed by a subsequent decrease, with the highest value achieved when the number of fluid units equaled the number of casualties.

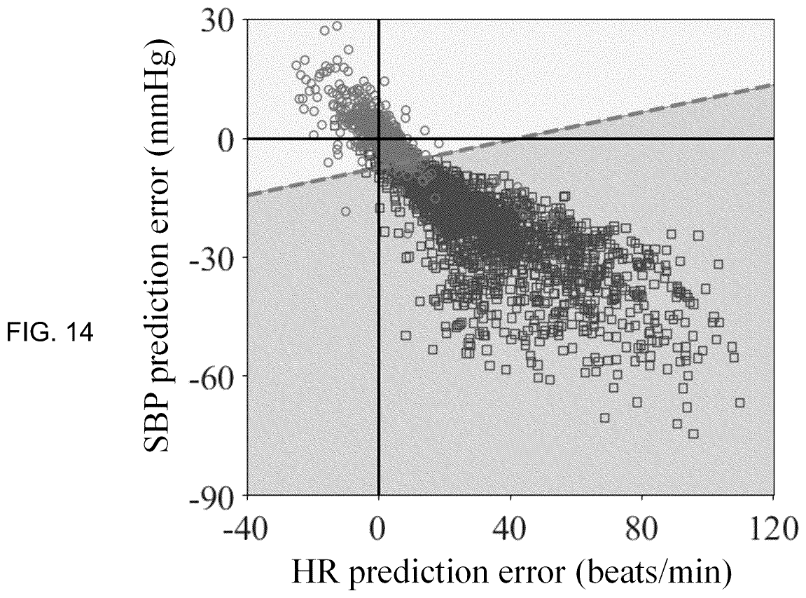

To verify if the AI model could detect uncontrolled non-compressible bleeding, 640 (4N_{F}) controlled bleeding trajectories and N_{N}=2,069 non-compressible bleeding trajectories were generated. FIG. 14 shows the classification results of the linear SVM in differentiating controlled versus non-compressible bleeding. The blue circles and red squares represent the prediction errors between the AI model and the CR model (CR minus AI) for HR and SBP at t₃ for the controlled and non-compressible bleeding scenarios, respectively. As expected for the controlled bleeding scenario, the mean prediction errors for both vital signs were close to zero. Most of the prediction errors ranged from −22 to 26 beats/minute for HR and from −24 to 27 mmHg for SBP (blue circles). In contrast, when non-compressible bleeding was present, it caused an increase in HR and a decrease in SBP, leading to corresponding changes in their prediction errors. The prediction errors ranged from −10 to 110 beats/minute for HR and from −72 to 9 mmHg for SBP (red squares). This significant difference in the prediction errors between the two scenarios serves as a potential indicator for detecting uncontrolled non-compressible bleeding.

The shaded areas in FIG. 14 represent the classified areas corresponding to the controlled and non-compressible bleeding regions, respectively, in the HR/SBP feature space. The red dashed line between the two areas denotes the decision boundary that separates the two scenarios. Because the decision boundary is neither horizontal nor vertical, both HR and SBP are essential for accurate classification. Quantitatively, the majority of the circles and squares fell within their respective regions, indicating that the SVM accurately classified the bulk of the trajectories associated with the two scenarios. Table 4 shows the quantitative classification results of monitored trajectories in the two scenarios. The model obtained an accuracy of 94% for the 640 controlled bleeding trajectories and an accuracy of 92% for the non-compressible bleeding trajectories, indicating the effectiveness of the AI model in detecting the presence of uncontrolled non-compressible bleeding.

TABLE 4
Classification results of the linear support vector machine algorithm
for monitored trajectories at the end of fluid resuscitation (t₃).

| | | Classified as | | Classifi- |
|---|---|---|---|---|
| | | | Non- | cation |
| | Number of | Controlled | compressible | accuracy |
| Scenario | trajectories | bleeding | bleeding | (%) |
| Controlled bleeding | 640 | 602 | 38 | 94 |
| Non-compressible | 2,049 | 165 | 1,904 | 92 |
| bleeding | | | | |

Classification results are shown for monitored trajectories in the following two scenarios: 1) when tourniquet application at t₁ controlled any and all bleeding (controlled bleeding) and 2) when tourniquet application at t₁ did not control all bleeding because there was additional non-compressible bleeding (non-compressible bleeding).

To assess the predictive performance of the model, its predictions were compared with those of the ground-truth CR model results for HR and SBP. The model takes 10 minutes of initial vital-sign data after tourniquet application to make personalized predictions of four different fluid-resuscitation treatments, ranging from no fluid up to 2 units of fluid in 60 minutes as illustrated in FIG. 3. The comparison of the predicted vital-sign trajectories against those of the CR data revealed low test errors (δₕ and δₛ), with 4.3 beats/minute (SD=0.7) for HR and 4.1 mmHg (SD=0.8) for SBP, indicating an overall accurate prediction of vital signs in response to different hemorrhage scenarios. The observed prediction errors were comparable to instrumental accuracy, and further attempts to reduce these errors could potentially lead to model overfitting and, hence, compromised model generalizability. While achieving zero errors would represent an ideal scenario where the model perfectly matched the CR model-based allocation, it is important to strike a balance between prediction accuracy and generalizability.

With the capability to prospectively evaluate treatment options based on limited initial vital-sign data and the model, near-optimal fluid resuscitation treatment can be selected before starting the fluid infusion. This allowed the model to construct a predictive allocation method that considered both the available resources and the number of casualties. To assess the performance of this allocation method, the evaluation conducted three analyses to compare it with the Vampire-based allocation method. Overall, the model allocations outperformed the Vampire-based allocations in all three analyses, based on different performance metrics.

A linear SVM was developed to distinguish between controlled bleeding and uncontrolled non-compressible bleeding, achieving high classification accuracies (>90%). These results highlight the effectiveness of the SVM in accurately distinguishing between these two bleeding scenarios, even as we considered a wide range of fractions (10%-50%) of uncontrolled non-compressible bleeding out of the total bleeding rate. As expected, as the fraction of non-compressible bleeding increased, it led to a more pronounced impact on HR elevation and SBP reduction, resulting in a relatively easier detection of non-compressible bleeding. Conversely, when the fraction of non-compressible bleeding was smaller, the corresponding changes in HR and SBP were less pronounced, making the detection task more challenging.

The example and alternative embodiments described above may be combined in a variety of ways with each other without departing from the invention.

As used above “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

The foregoing description describes different components of embodiments being “connected” to other components. These connections include physical connections, fluid connections, magnetic connections, flux connections, and other types of connections capable of transmitting and sensing physical phenomena between the components.

Although the present invention has been described in terms of particular embodiments, it is not limited to those embodiments. Alternative embodiments, examples, and modifications which would still be encompassed by the invention may be made by those skilled in the art, particularly in light of the foregoing teachings.

Those skilled in the art will appreciate that various adaptations and modifications of the embodiments described above can be configured without departing from the scope and spirit of the invention. Therefore, it is to be understood that, within the scope of the appended claims, the invention may be practiced other than as specifically described herein.

1. A method for allocating of resuscitation fluid to one or more casualties, the method comprising:
collecting multiple minutes of vital-sign data from vital-sign sensors attached to one casualty,
applying hemorrhage control to stop or largely stop the bleeding of the casualty,
inputting the collected vital-sign data into a model to generate a personalized predicted set of vital signs for the casualty after running multiple scenarios, wherein each scenario is associated with an amount and an infusion rate of resuscitation fluid to be provided to the casualty over a predetermined time period,
selecting the scenario that uses the least amount of resuscitation fluid to have, if possible, the casualty vital signs within healthy target ranges at the end of the predetermined time period while ensuring a largest number of casualties reach healthy target ranges, and
administrating resuscitation fluid to the casualty pursuant to the selected scenario; and
wherein the vital-sign data includes a heart rate and a systolic blood pressure, and
the infusion rate for resuscitation fluid is a third input into the model.

2. The method according to claim 1, wherein the predetermined time period is sixty minutes and the multiple scenarios include the following four scenarios for sixty minutes after application of the hemorrhage control:
providing the casualty no resuscitation fluid,
providing the casualty one unit of resuscitation fluid in either the first 30 minutes or the second 30 minutes, and
providing the casualty two units of resuscitation fluid with one unit in the first 30 minutes and the second unit in the second 30 minutes.

3. The method according to claim 1, wherein the resuscitation fluid is whole blood, blood products, saline, or crystalloids.

4. The method according to claim 1, wherein the vital sign data is collected for at least 10 minutes and the predetermined time period is sixty minutes.

5. The method according to claim 1, wherein the hemorrhage control is a tourniquet.

6. The method according to claim 1, wherein the model is a recurrent artificial neural network.

7. The method according to claim 6, wherein the model includes a series of layers between an input layer and an output layer, where each layer includes 128, 256, or 512 nodes.

8. The method according to claim 6, wherein the model includes an input layer, a first feedforward layer, a gated recurrent unit (GRU) layer, a second feedforward layer, and an output layer where the outputs of the first feedforward layer is a n-dimensional feature vector that is inputted into the GRU layer, the outputs of the GRU layer is a m-dimensional feature vector that is inputted into the GRU layer and the second feedforward layer, and the outputs of the second feedforward layer is a o-dimensional feature vector that in inputted in the output layer that provides the outputs for the model that are then inputted back into the model, and
wherein n, m, and o are equal to the number of nodes of the respective layer.

9. The method according to claim 1, wherein inputting the collected vital sign data into the model includes
receiving the heart rate at a given time (“HR(t)”), the fluid infusion rate at said given time (“uₜ(t)”), and the systolic blood pressure at said given time (“SBP(t)”) into an input layer that distributes the inputs to each node of a first feedforward layer, the first feedforward layer having a plurality of first feedforward layer nodes;
receiving a first dimensional feature vector produced by the first feedforward layer into a gated recurrent unit layer (“GRU”) having a plurality of GRU layer nodes for personalizing the prediction for the casualty based on the inputs into the GRU layer, and the GRU layer further receiving a second dimensional feature vector from the GRU layer previous time period on a second iteration of the method through the model;
receiving the second dimensional feature vector from the GRU layer into a second feedforward layer having a plurality of second feedforward layer nodes, where each second feedforward layer node receives the second dimensional feature vector;
receiving a third dimensional vector produced by the second feedforward layer into an output layer;
outputting a personalized predicted value of the heart rate at a future time (“HR(t+1)”) and a predicted value of systolic blood pressure at a future time (“SBP(t+1)”) from the output layer, and
wherein the method is repeatedly iterated for the predetermined time period, and the predetermined time period is sixty minutes, and
during a subsequent iteration of the method, the inputs sent to the nodes of the input layer and then to the first feedforward layer are the HR(t+1) and SBP(t+1) output from a prior iteration of the method.

10. The method according to claim 1, wherein the model is trained on vital signs generated by a cardio-respiratory mathematical model to simulate future heart rate and systolic blood pressure based on inputted heart rates, systolic blood pressures, and infusion rates.

11. The method according to claim 1, wherein the model includes at least one hidden state, and utilizes measured vital sign data to update the at least one hidden state to personalize the model for the casualty, and starting at application of hemorrhage control, the model continuously predicts the vital sign data for each subsequent minute using the infusion rate associated with one of the scenarios and feeding back into the model the calculated vital signs until the model predicts the vital sign data at the end of the predetermined time period to allow for a comparison between the multiple scenarios.

12. A method for allocating of resuscitation fluid to one or more casualties, the method comprising:
for each casualty,
collecting multiple minutes of vital-sign data from vital-sign sensors attached to the casualty, where the vital-sign data includes a heart rate and a systolic blood pressure,
applying hemorrhage control to stop or largely stop the bleeding of the casualty,
inputting the collected vital-sign data into a model to generate a personalized predicted set of vital signs for the casualty after running multiple scenarios, wherein each scenario is associated with an amount and an infusion rate of resuscitation fluid to be provided to the casualty over a predetermined time period,
displaying to a dashboard a recommended scenario that uses the least amount of resuscitation fluid to have, if possible, the casualty vital signs within healthy target ranges at the end of the predetermined time period, and
administrating resuscitation fluid to the casualty pursuant to the recommended scenario; and
wherein the dashboard that includes information for each casualty including when to administer resuscitation fluid to each casualty considering a supply level of resuscitation fluid.

13. A system for providing a recommendation for use of resuscitation fluid in hemorrhage treatment for multiple casualties, the system comprising:
vital sign sensors configured to be attached to each casualty, the vital sign sensors include a heart rate monitor and a systolic blood pressure sensor;
a memory;
a processor having a model trained on hemorrhage control and resuscitation fluid recovery scenarios, the model configured to provide a recommendation regarding use of resuscitation fluid once hemorrhage control has begun on the casualty, the processor in communication with the vital sign sensors and configured to receive vital sign data from same for storage in the memory, the processor running an instance of the model for each casualty and providing the output to a dashboard; and
a display in communication with the processor and configured to provide recommendations and/or vital sign data to an individual treating each casualty via the dashboard.

14. The system according to claim 13, wherein the model predicts vital signs of each casualty under a plurality of scenarios including the following four scenarios for sixty minutes after application of the tourniquet:
providing the casualty no resuscitation fluid,
providing the casualty one unit of resuscitation fluid in either the first 30 minutes or the second 30 minutes, and
providing the casualty two units of resuscitation fluid with one unit in the first 30 minutes and the second unit in the second 30 minutes of the sixty minutes.

15. The system according to claim 13, wherein the model is a recurrent neural network.

16. The system according to claim 15, wherein the model includes a plurality of layers between an input layer and an output layer, each layer having 128, 256, or 512 nodes.

17. The system according to claim 15, wherein the model
includes an input layer, a first feedforward layer, a GRU layer, a second feedforward layer, and an output layer where the outputs of the first feedforward layer is a n-dimensional feature vector that is inputted into the GRU layer, the outputs of the GRU layer is a m-dimensional feature vector that is inputted into the GRU layer and the second feedforward layer, the outputs of the second feedforward layer is a o-dimensional feature vector that is inputted into the output layer that provides the outputs for the model that are then inputs back into the model, and
wherein n, m, and o are equal to the number of nodes of the respective layer.

18. The system according to claim 13, wherein the model includes
an input layer, a first feedforward layer, the first feedforward layer having a plurality of first feedforward layer nodes, where each first feed feedforward layer node receives the heart rate at a given time (“HR(t)”), the fluid infusion rate at said given time (“uₜ(t)”), and the systolic blood pressure at said given time (“SBP(t)”);
a gated recurrent unit layer (“GRU”) having a plurality of GRU layer nodes for personalizing the prediction for the casualty based on a first dimensional feature vector produced by the first feedforward layer, and the GRU layer further receiving a second dimensional feature vector from the GRU layer previous time period on a second iteration of the model;
a second feedforward layer having a plurality of second feedforward layer nodes, where each second feedforward layer node receives the second dimensional feature vector, the second feedforward layer outputs to an output layer that outputs a predicted value of the heart rate at a future time (“HR(t+1)”) and a predicted value of systolic blood pressure at a future time (“SBP(t+1)”), which then are provided as inputs back into the model, and
wherein the model is repeatedly iterated for a predetermined time period, and the predetermined time period is sixty minutes.

19. The system according to claim 13, wherein the model is trained on vital signs generated by a cardio-respiratory mathematical model to simulate future heart rates and systolic blood pressure based on inputted heart rates, systolic blood pressures, and infusion rates.

20. The system according to claim 13, wherein the model utilizes measured vital-sign data to update at least one hidden state to personalize the model for the casualty, and starting at application of hemorrhage control, the model continuously predicts the vital-sign data for each subsequent minute using the infusion rate of the associated scenario and feeding back into the model the calculated vital-signs until the model reaches a desired measuring time to allow for a comparison between the multiple scenarios.
